(19) United States
(12) Patent Application Publication
(10) Pub. No.: US 2007/0231869 A1
(43) Pub. Date: Oct. 4, 2007

(54) Fermentation Process, Starter Culture and Growth Medium

(75) Inventors: Marie Holmgren, Vilhelmina (SE); Anita Sellstedt, Umea (SE)

Correspondence Address: MORRISON & FOERSTER LLP, 425 MARKET STREET, SAN FRANCISCO, CA, 94105-2482, US

(73) Assignee: SWETREE TECHNOLOGIES, Umea (SE)

(21) Appl. No.: 10/581,507

(22) PCT Filed: Dec. 1, 2004
(86) PCT No.: PCT/SE04/01786

§ 371 Date: Apr. 25, 2007

(30) Foreign Application Priority Data
Dec. 1, 2003 (SE) 0303225-7
Jun. 4, 2004 (SE) 0401447-8

(51) Int. Cl. C12P 7/06

(52) U.S. Cl. 435/165; 435/254.11

(57) ABSTRACT

Ethanol production from biomass can be rendered more effective by the use of at least one fungus or a mix of fungi capable of fermenting pentose compounds, or both pentose as well as hexose compounds. Preferably said at least one fungus is a fungus belonging to the species Chalara sp., optionally used in combination with a second fungus belonging to the species Trametes sp. Preferably said fungus or fungi is/are used in combination with other fermenting microorganisms, such as a yeast, e.g. Saccaromyces cerevisiae.

### FIELD OF THE INVENTION

The present invention relates to the field of biotechnology, and in particular to ethanol production through the fermentation of one or more organic starting materials. Specifically, the invention relates to a process for ethanol production wherein at least one fungus capable of metabolizing 5-carbon compounds, or a mix of fungi, is used to produce ethanol and/or to enhance the ethanol yield.

### BACKGROUND OF THE INVENTION

The use of fossil fuels has contributed to environmental problems, including the increased emission of CO₂, a gas implicated in global warming. A significant increase in atmospheric CO₂ concentration has been recorded during the past 350 years. The use of renewable resources as an alternative to fossil fuels has been under investigation for many years. Compared to the increasing use of fossil fuels, which is a limited resource, the production of ethanol from biomass offers a promising alternative.

Ethanol can be regarded as more environmentally friendly than fossil fuels. Considerable research efforts are therefore conducted to find economical ways of producing ethanol from renewable raw materials. Ethanol from biomass is produced through fermentation of sugar and polysaccharide containing materials. Sugarcane or maize are feed stocks of interest, but the raw material cost would then constitute a great part of the total ethanol cost. It is important to be able to use low cost raw material such a lignocellulosic materials e.g. fast growing trees, grass, waste products such as agricultural and forestry residues, in order to make ethanol competitive with fossil fuels. Process improvements and new technology in this field are therefore of considerable commercial and environmental interest. The use of lignocellulosic materials is apparently very advantageous, because it is the most abundant renewable organic material in the biosphere.

Most plant materials can be commonly described as lignocellulosic biomass. Lignocellulose is composed of three major constituents i.e. cellulose (35-50%), hemi-cellulose (20-35%) and lignin. Minor constituents of lignocellulose are ash, phenolics, extractives and trace residues. The major compound cellulose is a linear polymer of D-glucose linked together by β-1,4-glucosodic bonds to create a water-insoluble polysaccharide. The cellulose molecules are organized in elementary fibrils associated with hydrogen and van der Waals bonds, forming a very rigid structure of micro fibrils. The micro fibril contains regions of amorphous structure that are susceptible to hydrolysis. Other important polysaccharides are the hemicelluloses, branched polymers of different monomeric sugars. Hemicelluloses link through hydrogen bonds to cellulose and through covalent bonds to lignin. Another important compound in wood is lignin, which is one of the most abundant substances in the plant world. Lignin forms a composite structure with the cellulose, significantly increasing the mechanical strength of the wood. Relatively few microorganisms can degrade lignin effectively, which makes wood a very durable material.

The production of ethanol from fermentation of sugars or polysaccharides in biomass is of considerable economic and environmental interest. Cellulose and the hemicelluloses in biomass all consist of long chains of sugar molecules. In order to enable the production of ethanol, the sugar molecules needs to be separated by hydrolysis of the long chains in which they are stored.

### PRIOR ART

Ethanol production from industrial lignocellulose material has been the focus of considerable research. One approach is to modify existing pre-treatment steps, or to introduce new, more effective pre-treatment steps. Numerous reports have been published, dealing with the pre-treatment of biomass and how to avoid inhibitors that are a by-product of such pre-treatments.

Another approach lies in the genetic modification of the microorganisms used, i.e. mainly yeast. Microorganisms that ferment the glucose component in the cellulose to ethanol are well known in the art. However, the availability of microorganisms that efficiently ferment the 5-carbon sugars, xylose and arabinose, in the hemicelluloses to ethanol has been one of the main obstacles for improved ethanol production from biomass.

Recently, Pretorius et al. (Food Technol. Biotechnol. 41:3-10, 2003) focused on the need of modifying Saccharomyces cerevisiae for more efficient use of the pentoses in wood and other hemicellulosic materials for ethanol production. However, the genetically modified yeast strains described tend to be less efficient. Patil et al. (Enzyme Microb. Technol., 1990, vol 12, 141-148) suggest the addition of fungal mycelium to accelerate ethanol production from cane molasses batch fermentation using Saccharomyces cerevisiae. The following fungi were investigated: Penicillium chrysogenum, Aspergillus oryzae, Sclerotium rolfsii, Sporotrichum puliverulentum, Aspergillus niger, Rhizopus nigricans, Neurospora sitophilia, Fusarium tricinctum, and Trichoderma reesei. The authors conclude that a mycelium supplement with as many as 10 different fungal species could accelerate ethanol production, and advocate the use of waste mycelium from the antibiotic industry. Trace amounts of antibiotics present in the mycelium are believed to be beneficial in the removal of bacterial contamination during fermentation.

There remains a need for alternative approaches to enhanced ethanol fermentation, and in particular industrially applicable and economically competitive processes. It is of particular interest to be able to utilize also the 5-carbon sugars, such as xylose and arabinose, in hemicellulose. An important aim of the present invention is to make available such processes without resorting to genetic modification of the microorganisms involved.

Further aims underlying the invention, and advantages associated with the invention, will be evident to a skilled person from the description and examples.

### SUMMARY OF THE INVENTION

The present invention makes available an improved process for the production of ethanol through fermentation of one or more organic starting materials, or for facilitating and/or contributing to such fermentation, characterized by the features enumerated in the claims, incorporated herein by reference.

The invention also makes available a starter culture, as well as a growth medium, as defined in the claims, incorporated herein by reference.

Further, the invention presents a growth medium for a fungus used in the inventive process.

### SHORT DESCRIPTION OF THE DRAWINGS

The invention will be described in closer detail in the following description, non-limiting examples, and attached drawings, in which:

FIG. 1 shows the growth of a mixture of fungi for 65 h on xylose as the main carbon source in SeHo medium.

FIG. 2 shows the growth of a mixture of fungi for 69.5 h on mannose as the main carbon source in SeHo medium.

FIG. 3 shows the growth of a mixture of fungi for 66 h on galactose as the main carbon source in SeHo medium.

FIG. 4 shows the growth of a mixture of fungi for 50 h in a starch-containing medium.

FIG. 5 shows the growth of a mixture of fungi for 115 h in an experimental acid hydrolysate (pulp waste).

FIG. 6 shows the accumulated ethanol production in wood hydrolysate with different amounts of yeast and microorganisms. 1=0.05 g mixed fungi, 0.02 g S. cerevisiae; 2=0.025 g mixed fungi, 0.01 g S. cerevisiae; 3=0.2 g mixed fungi, 0.08 g S. cerevisiae; 4=0.05 g mixed fungi, 0.04 g S. cerevisiae; 5=0.10 g mixed fungi, 0.02 g S. cerevisiae.

FIG. 7 shows the accumulated ethanol production in an experimental acid hydrolysate with 0.2 g mixed fungi and 0.08 g S. cerevisiae (g fresh weight (FW)/I).

FIG. 8 shows the ethanol production in a wood hydrolysate (WH) using mixed fungi 0.2 g (C.P.), Chalara parvispora CBS strain 983.73 (983), and C. parvispora CBS strain 385.94 (385).

### DETAILED DESCRIPTION OF THE INVENTION

### Process for the Production of Ethanol

The present invention relates to a process for enhanced production of ethanol from biomass. It is based on the surprising discovery of a group of microorganisms capable of fermenting pentoses, and even capable of fermenting both pentose and hexose compounds, as well as their utility in ethanol production.

More specifically, the present invention makes available a process for the production of ethanol through fermentation of organic starting materials, wherein at least one fungus, or a mix of fungi, capable of metabolizing pentose compounds is used. Said at least one fungus is optionally also capable of fermenting hexose compounds. Said at least one fungus is preferably chosen among soft rot fungi, brown rot fungi, black rot fungi and white rot fungi, more preferably chosen among Chalara sp., Trametes sp., Trichoderma sp., Thielavia sp., Postia sp., Gloeophyllum sp., Phanerochaete sp., Xylaria sp., or a combination thereof.

The present inventors surprisingly found one particular fungus, and showed that this has utility in ethanol production. This was identified as Chalara parvispora, a species growing well on 5-carbon sugars as well as 6-carbon sugars. Other fungi, also verified to have the capability to produce ethanol, are soft rot fungi, here exemplified by Trichoderma viride and Thielavia terrestris; brown rot fungi, exemplified by Postia placenta and Gloeophyllum trabeum; and white rot fungi, exemplified by Phanerochaete chrysosporium and Trametes versicolor.

According to one embodiment of the invention, Chalara parvispora is used for the production of ethanol through fermentation of organic starting materials. When used in combination with one or more fungi, the preferred second fungus is Trametes versicolor.

The most frequently used microorganism in hexose fermentation is S. cerevisiae, also known as baker's yeast. S. cerevisiae is capable of producing ethanol from glucose and mannose if the concentration of sugars is high or when the yeast is grown under anaerobic or semi-anaerobic conditions. Thus, according to one embodiment of the present invention, Chalara parvispora, alone or as part of a mixture of fungi, is used in combination with at least one type of yeast. The yeast may belong to a species of Saccharomyces, preferably S. cerevisiae. Other species of yeast that can be used are, for example, species belonging to Candida sp., such as C. shehateae, species belonging to Pichia sp. such as P. bovis, and species belonging to Clavispora sp.

The fungus can also be used in combination with other ethanol-producing microorganisms to optimize substrate utilization, both 5-carbon metabolizing microorganisms and/or 6-carbon metabolizing microorganisms. For example, there are strains of fungi (e.g. Fusarium, Mucor, Monilia and Paecilomyces) that are able to produce ethanol from D-xylose, but they are considered to produce less ethanol than yeast. It is contemplated that also genetically modified microorganisms can be used, although one aim of the present inventors was to identify useful, naturally occurring microorganisms, in order to reduce the need for genetically modified microorganisms.

It is also contemplated that enzymes are added to the process in order to facilitate the degradation of substrates and to enhance ethanol production. For example, cellulase can be added to degrade cellulose and hemicellulase to degrade hemicellulose. There are numerous examples of additional enzymes that can be used to convert substrates to enhance ethanol production, for example aldose reductase and xylitol reductase, in order to facilitate the conversion of pentoses to hexoses. A skilled person can easily, by routine steps and without undue experimentation, chose suitable enzymes and determined their dosage.

It is further contemplated that other means of facilitating the degradation of substrates can be used in the process, examples including, but not limited to mechanical disruption, ultrasonication, or steam and high-pressure pre-treatments.

In the process according to the invention said at least one fungus and said yeast are multiplied separately before use in a bioreactor. The fungus can be added to the organic material prior to the yeast or substantially simultaneously with the addition of the yeast. When the yeast is S. cerevisiae, it is preferably cultured for about 24 h before addition to the biomass. The fungi mix is grown for about 24-48 h, i.e. until reaching log phase, before addition to the starting material. An amount of about 0.05 to 0.2 g cells (fresh weight) was added per liter.

Regarding the use of yeast, the choice of yeast, and the handling thereof, a skilled person will be able to use known processes or can easily adapt these to the use according to the present invention.

In process of the invention the pH of the starting materials is adjusted to the range of about pH 5-6.5, preferably 5.5-6.2, and most preferably about pH 6. The pH may be adjusted by the addition of appropriate amounts of an alkali or an acid according to well-known procedures. The fermentation is performed in a temperature interval of about 24 to 36° C., preferably about 26 to about 29° C., more preferably at about 27° C. Other fermentation coditions, such as agitation, addition of co-substrates, nutrients, time and degree of anaerobiosis can be optimized according to the nature of the starting material and the fermenting microorganism(s) used. A skilled person can further adjust the fermentation conditions to the use of different starting materials.

The process according to the invention can be performed as a batch fermentation, wherein the microorganisms are killed or otherwise discarded after the fermentation. In another embodiment of the invention the fermentation process is performed as a continuous or semi-continuous process, where starting materials and/or nutrients are added during fermentation. To retain the microorganisms in the bioreactor they can be separated from the solids by any suitable means, for example sedimentation or centrifugation.

To obtain the ethanol after the fermentation, the biomass first needs to be separated from the fluids by means such as centrifugation or sedimentation. Subsequently, the ethanol can be separated from the biomass by any conventional method, such as distillation, membrane separation, enzyme process and gasification. Such methods are known per se and do not constitute part of the present invention.

According to the invention the starting material can be any organic material that can be fermented for the production of ethanol. The ethanol can be produced from any lignocellulosic biomass. Relevant starting material include wooden or non-wood plant material, e.g. stem, stalk, shrub, hulls, foliage, fibre, shell, root, straw, hay, grass, reed etc. Sources of wood can be any species of softwood or hardwood trees. Sources of straw include in particular cereals and cereal grasses, such as oat, wheat, barley, rye, maize and rice. Additional sources can be root-crops, such as beets and tubers. The above examples are intended for illustrative purposes only, and are not limiting the scope of the invention.

Further example of starting materials include waste or by-products from forestry, such as wood chips, saw dust etc; as well as solid or liquid effluents or by-products from pulp and paper industry, such as wood hydrolysates of different origin and in different states of processing; paper waste, such as waste from the production or recycling of newspapers, magazines, photocopying and computer printer papers and paper based packaging. Preferred starting materials include spent liquor or waste liquor from pulping, such as acidic waste liquor, acidic sulphite waste liquor, neutralized waste liquor etc, including combinations thereof, such as mixed waste streams.

Further example of starting materials include solid or liquid effluents or by-products from food and feed industry, for example effluents or by-products containing cellulose, hemicellulose, sugar or starch; solid or liquid waste or by-products from agriculture; by-products from gardening such as garden refuse or other waste or by-product streams or their components comprising compounds that can be fermented.

The starting material may be any of the above-mentioned materials in treated or untreated from. A skilled person can implement possibly necessary pretreatment steps without inventive effort and without undue experimentation.

Starter Culture

The present invention also relates to a starter culture for use in the inventive process. The starter culture comprises at least one fungus, or a mixture of fungi, capable of metabolizing pentose compounds. Preferably said at least one fungus is also capable of metabolizing hexose compounds. In one embodiment the fungus or fungi is/are chosen among brown rot fungi, soft rot fungi, and white rot fungi or a combination thereof, for the manufacture of a starter culture for the use in the production of ethanol.

Preferably said fungus is chosen among Chalara sp., Trametes sp., Trichoderma sp., Thielavia sp., Postia sp., Gloeophyllum sp., Phanerochaete sp., Xylaria sp., or a combination thereof. More preferably, said fungus is chosen among Chalara parvispora, Trametes versicolor, Trichoderma viride, Thielavia terrestris, Postia placenta, Gloeophyllum trabeum, Phanerochaete chrysosporium, or a combination thereof.

Most preferably, said at least one fungus is Chalara parvispora. When used in combination with one or more fungi, the preferred second fungus is Trametes versicolor.

The fungus or fungi can also be used in combination with other microorganisms, such as fungi, yeasts and bacteria. Preferably the fungus is used in combination with a yeast belonging to the species Saccharomyces, such as S. cerevisiae. Other species of yeast that can be used are, for example, species belonging to Candida sp., such as C. shehateae, species belonging to Pichia sp. such as P. bovis, and species belonging to Clavispora sp.

The starter culture may be used in combination with other microorganisms, such as other fungi, yeasts and bacteria.

Growth Medium

The present invention also relates to a growth medium for a fungus used in the inventive process. The medium is tentatively called SeHo-medium. The composition is given in Table 1 (the concentration of the components are given as approximate values):

TABLE 1
Growth medium

| Component | Final concentration (gram/litre) |
|---|---|
| CaCl₂ 2H₂O | 0.0130 |
| MgSO₄ 7H₂O | 0.030 |
| K₂HPO₄ | 0.95 |
| NaH₂PO₄ 2H₂O | 0.80 |
| D-xylose | 25 |
| D-mannose | 25 |
| D-galactose | 25 |
| NH₄Cl | 0.5 |
| Salts | 0.040 |

The growth medium of Table 1 may further comprise starch at a final concentration of about 25 g/l.

Use of a Fungus or Mix of Fungi

The present invention also relates to the use of at least one fungus, or a mix of fungi chosen among brown rot fungi, soft rot fungi, black rot fungi, and white rot fungi or a combination thereof, for the fermentation of an organic starting material in the production of ethanol, or for facilitating and/or contributing to such fermentation.

Preferably said at least one fungus is chosen among Chalara sp., Trametes sp., Trichoderma sp., Thielavia sp., Postia sp., Gloeophyllum sp., Phanerochaete sp., Xylaria sp., or a combination thereof. More preferably, said fungus is chosen among Chalara parvispora, Trametes versicolor, Trichoderma viride, Thielavia terrestris, Postia placenta, Gloeophyllum trabeum, Phanerochaete chrysosporium, or a combination thereof.

The fungus or fungi can also be used in combination with other microorganisms, such as fungi, yeasts and bacteria. Preferably the fungus is used in combination with a yeast belonging to the species Saccharomyces, such as S. cerevisiae. Other species of yeast that can be used are, for example, species belonging to Candida sp., such as C. shehateae, species belonging to Pichia sp. such as P. bovis, and species belonging to Clavispora sp.

The starting material can be any of the above-mentioned starting materials. Said at least one fungus can also be used in combination with other microorganisms, such as fungi, yeasts and bacteria. Preferably the fungus is used in combination with a yeast belonging to the species Saccharomyces, such as S. cerevisiae.

According to another embodiment, the invention relates to the use of at least one fungus chosen among brown rot fungi, soft rot fungi, and white rot fungi or a combination thereof, for the manufacture of a starter culture for the use in the production of ethanol, or for facilitating and/or contributing to such fermentation.

Preferably said fungus is chosen among Chalara sp., Trametes sp., Trichoderma sp., Thielavia sp., Postia sp., Gloeophyllum sp., Phanerochaete sp., Xylaria sp., or a combination thereof. More preferably, said fungus is chosen among Chalara parvispora, Trametes versicolor, Trichoderma viride, Thielavia terrestris, Postia placenta, Gloeophyllum trabeum, Phanerochaete chrysosporium, or a combination thereof.

Most preferably, said at least one fungus is Chalara parvispora. When used in combination with one or more fungi, the preferred second fungus is Trametes versicolor.

The fungus or fungi can also be used in combination with other microorganisms, such as fungi, yeasts and bacteria. Preferably the fungus is used in combination with a yeast belonging to the species Saccharomyces, such as S. cerevisiae. Other species of yeast that can be used are, for example, species belonging to Candida sp., such as C. shehateae, species belonging to Pichia sp. such as P. bovis, and species belonging to Clavispora sp.

### Advantages of the Invention

The present inventors have shown that ethanol production in batch cultures from biomass can be greatly increased compared to fermentation using only the well known Saccharomyces cerevisiae (baker's yeast). Thus, this invention is of high economic and environmental interest.

One important advantage of the invention is that the ethanol production can be optimized with only minor changes in existing processes, meaning e.g. that there is no expense for rebuilding existing bioreactors. Consequently, the cost for ethanol production can be significantly reduced in existing bioreactors. If the cost for ethanol production is reduced, the use of ethanol as a replacement for fossil fuels will be more attractive.

Another advantage is that the present invention makes it possible to use low cost feed, such as different types of waste, previously considered difficult or even impossible to utilize in the production of ethanol.

It can also be held to be a significant advantage that the improved fermentation can be achieved without resorting to genetic modification of the microorganisms.

Further aspects of the invention, and the advantages associated therewith, will be evident to a skilled person upon study of the description, examples and claims.

The present invention will now be described in the following non-limiting examples.

### EXAMPLES

### Example 1

### Growth of a Mixture of Fungi in Growth Medium Supplemented with Different Carbon Sources

In each experiment, fifteen 100 ml bottles were used. The cultures were inoculated with 0.05 g fresh weight (FW) fungi/l growth medium (see above) and grown at 27° C. for 50 to 65 h (se below) and were randomly weighed (wet weight), three bottles at four or five different points of time (in addition to time zero).

The growth of a mixture of fungi on different carbon sources was investigated by supplementing the medium with xylose 25 g/l, mannose 25 g/l, galactose 25 g/l and starch 25 g/l, respectively. The growth of a mixture of fungi in a newly designed hydrolysate was also investigated and the growth recorded as described above. The cultures supplemented with xylose were weighed 17, 24, 41, 48, and 65 h after inoculation. The cultures supplemented with mannose were weighed 14.5, 38.5, 45.5, 62.5, and 69.5 h after inoculation. The cultures supplemented with galactose were weighed 18, 24, 43, 48, and 66 h after inoculation. The cultures supplemented with starch were weighed 17, 24, 36 and 50 h after inoculation. The cultures grown in wood hydrolysate were weighed 19, 43, 67, 91, and 115 h after inoculation.

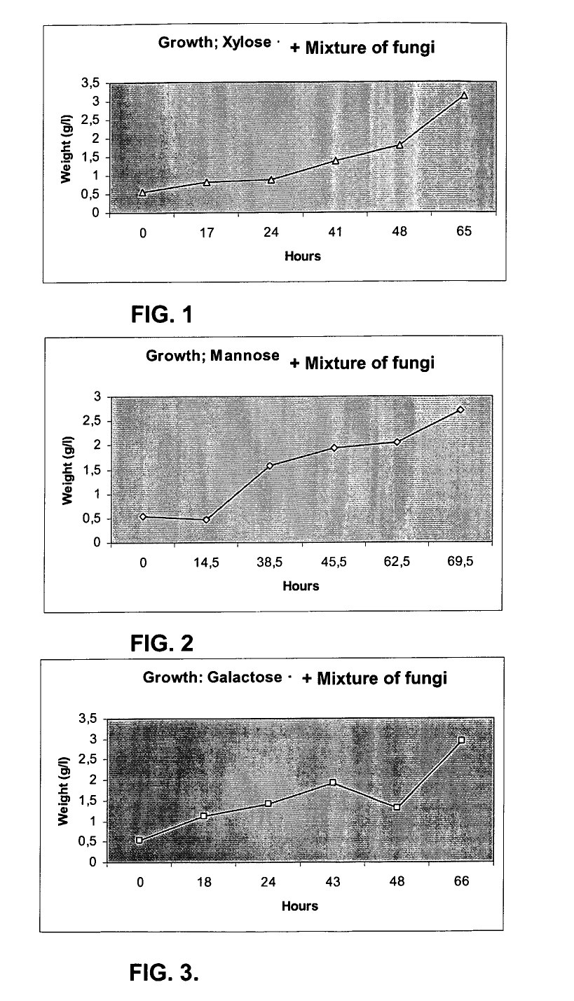

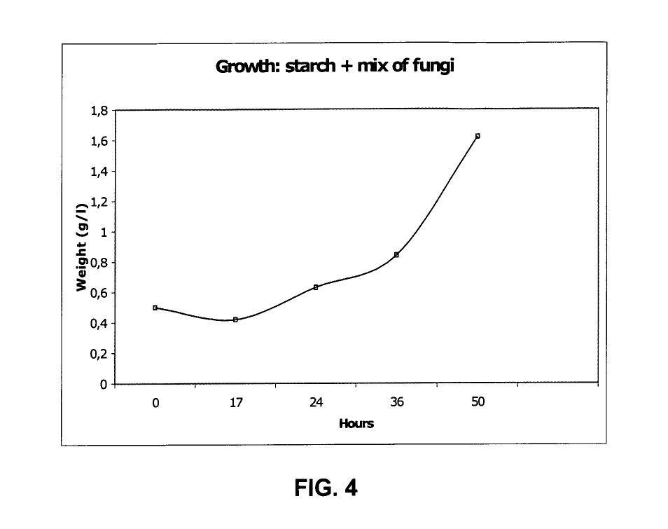

The results are summarized in the diagrams attached as FIGS. 1 through 5. The diagrams in FIGS. 1 through 4 show that the mixture of fungi grows equally well on xylose, mannose, galactose and starch as the carbon source, respectively. It is thus shown that the mixture of fungi is able to ferment both 5-carbon and 6-carbon compounds.

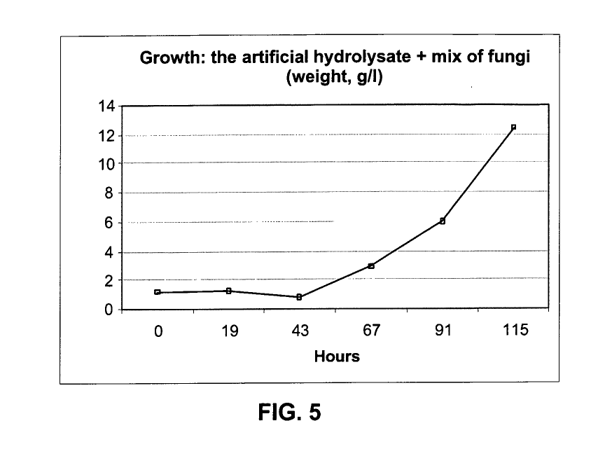

The mixture of fungi was also able to grow in a wood hydrolysate. The mixture exhibited an even better growth in a hydrolysate (FIG. 5) than that registered for any single carbon source. The mixture has been shown to comprise fungi belonging to Chalara sp., Trametes sp., and Xylaria sp. Subsequently; these fungi have been identified inter alia as Chalara parvispora and Trametes versicolor.

### Example 2

### Ethanol Production in Wood Hydrolysate Using Different Amounts of Microorganisms

Ethanol production in wood hydrolysate was investigated using different amounts of yeast (S. cerevisiae) and a mixture of fungi (see Table 2).

The yeast S. cerevisiae and the mixture of fungi were grown separately in YEP- and SeHo-medium for 24 and 48 h, respectively. YEP is a medium based on YPD, a complex medium for routine growth, but is without dextrose and can be used as a base for making media with alternate carbon source. At the start of the ethanol production experiments, different amounts of the microorganisms (see Table 2) were introduced into 100 ml flasks containing a wood hydrolysate (pH set to 6.0). The flasks were argonised to obtain an anaerobic atmosphere and subsequently incubated at 27° C. for 113 h under agitation (150 rpm/h).

TABLE 2
Amount of microorganisms used for production of ethanol in
wood hydrolysate

| | Amount of S. cerevisiae | Amount of mixture of |
|---|---|---|
| Sample | (g) | fungi (g) |
| 1 | 0.02 | 0.05 |
| 2 | 0.01 | 0.025 |
| 3 | 0.08 | 0.2 |
| 4 | 0.04 | 0.05 |
| 5 | 0.05 | 0.10 |

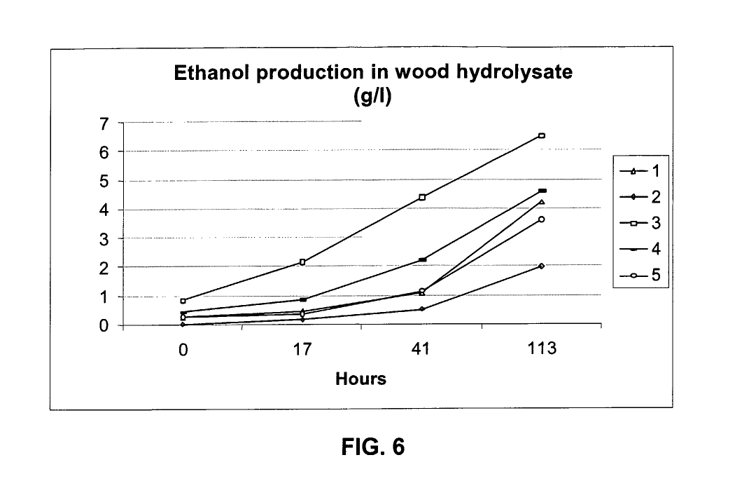

The result can be seen in FIG. 6. The highest amount of ethanol produced was recorded for sample 3, inoculated with S. cerevisiae (0.08 g/l) and the mixture of fungi (0.2 g/l).

However, the yeast was not grown in pulp waste before start of the experiment. It is contemplated that adaptation of the yeast would further improve the results.

### Example 3

### Ethanol Production from Lignocellulose in an Experimental Hydrolysate

In this experiment ethanol production in an experimental hydrolysate was investigated using S. cerevisiae and a mixture of fungi.

Three bottles with 100 ml of an experimental hydrolysate (See Table 3), containing S. cerevisiae and a mixture of fungi, was argonised to anaerobiosis. Samples of accumulated ethanol production was taken after 19, 43, 66, 91 and 137 h and analysed by gas chromatography.

TABLE 3
Components of the experimental hydrolysate

| | |
|---|---|
| Xylose | 11 g/l |
| Mannose | 27 g/l |
| Glucose | 9.7 g/l |
| Galactose | 4.7 g/l |
| Arabinose | 0.69 g/l |
| Salts | 0.040 g/l |
| Phosphate buffer | 1.75 g/l |
| NH₄Cl | 0.5 g/l |
| Sterilized water up to 1 l | |

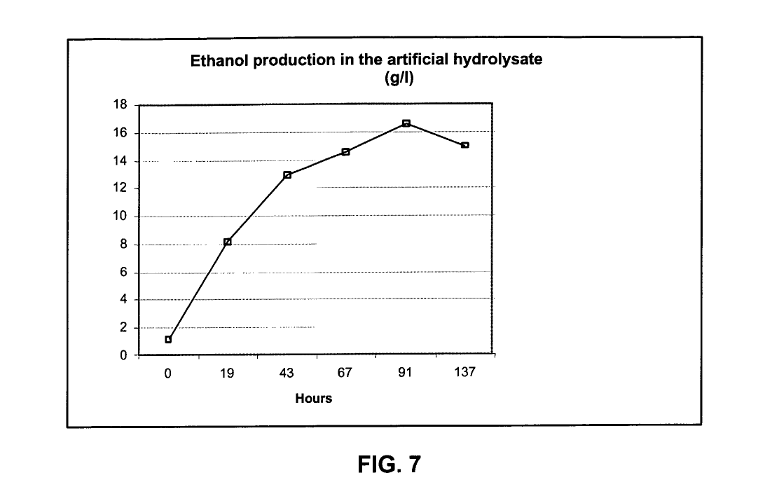

The results are shown in FIG. 7. A clear increase in ethanol production was observed, compared to the results shown in FIG. 6, i.e. about 17 g ethanol/l compared to 6.8 g ethanol/l. The increase is believed to be at least partially due to the fact that less inhibitory substances are present in the medium, which contains only pure chemicals.

### Example 4

### Ethanol Production from Lignocellulose in Pulp Waste

In this experiment ethanol production from lignocellulose in pulp waste was investigated. Ethanol production in pulp waste with S. cerevisiae was compared to ethanol production from pulp waste with both S. cerevisiae and a mixture of fungi.

Ten bottles each containing 100 ml of pulp waste (obtained from a Swedish pulp and paper mill) was used. Before the start of the experiment, the mixture of fungi was grown in wood hydrolysate for 24 h, in order for the fungi to adapt to the pulp waste. Three bottles were inoculated with S. cerevisiae only and 3 bottles were inoculated with both S. cerevisiae and a mixture of fungi. The four remaining bottles were used as controls and contained YEP-medium and both the microorganisms. All bottles were put under anaerobic atmosphere by flushing with argon and thereafter kept shaking (15-20 rpm/min) at 27° C. The amount of produced ethanol was measured after 164.8 h using gas chromatography

The results are shown in Table 4. The amount of ethanol produced by S. cerevisiae alone in pulp waste was 5.84 g/l, whereas 23.43 g/l was produced by S. cerevisiae and a mixture of fungi in combination in pulp waste. Thus, a nearly 4-fold increase in ethanol production was achieved by the addition of a mixture of fungi. This experiment showed that ethanol production in lignocellulose waste from the pulp industry can be significantly increased by the use of an additional microorganism, here exemplified by a mixture of fungi, shown to comprise C. parvispora. During the priority year, this mixture has been shown to comprise fungi belonging to Chalara sp., Trametes sp., and Xylaria sp. Subsequently; these fungi have been identified inter alia as Chalara parvispora and Trametes versicolor.

In addition, the results show that the a mixture of fungi can be “trained” to tolerate the pulp waste since the ethanol production in this experiment was higher than in the designed hydrolysate. The production can probably be further improved by the use of agents adsorbing the rest products of phenols and extractives.

TABLE 4
Amount of ethanol produced from lignocellulose in pulp waste

| | Amount ethanol produced |
|---|---|
| Microorganism used | (g/l) |
| S. cerevisiae + pulp waste | 4.88 +/− 0.85 |
| S. cerevisiae + a mixture of fungi + YEP | 17.98 +/− 0.39 |
| medium | |
| S. cerevisiae + a mixture of fungi + pulp | 23.10 +/− 0.39 |
| waste | |

### Example 5

### Ethanol Production from C. parvispora

Different C. parvispora strains (CBS strains 983.73 and 385.94) were grown in SeHo-medium for 48 h. At the start of the ethanol production experiments, 0.2 g FW of the microorganisms was introduced into 10 ml tubes containing wood hydrolysate (pH 6.0). The tubes were argonised to obtain anaerobic atmosphere and thereafter kept at a constant temperature of 27° C. and agitated (150 rpm/h). The experiment was run for 118 h.

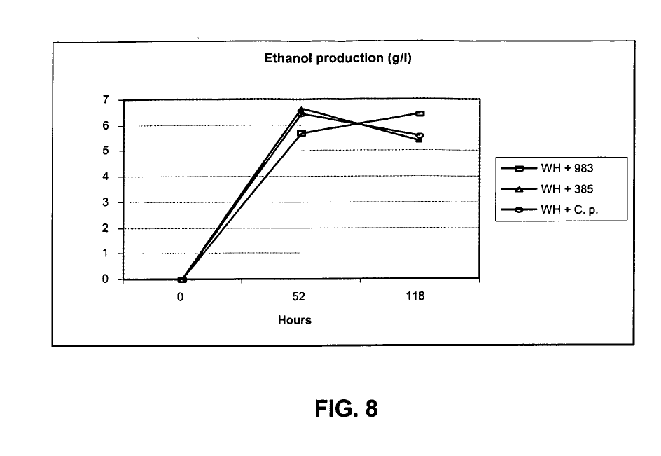

The results as shown in FIG. 8 clearly indicate that C. parvispora strains 983 and 385, as well as mixture (C.P.) (characterization not completed yet) have the capability of producing ethanol in a wood hydrolysate (WH).

During the priority year, this mixture has been shown to comprise fungi belonging to Chalara sp., Trametes sp., and Xylaria sp. Subsequently; these fungi have been identified inter alia as Chalara parvispora and Trametes versicolor.

### Example 6

### Ethanol Production by Different Rot Fungi from Lignocellulose in Pulp Waste

Fermentation tests were conducted in 10 ml tubes in order to detect occurrence of ethanol production in seven different rot fungi. Before the start of the experiment, the fungi were grown in a xylose-medium for 7-14 days. At the start of the experiment, 0.02 g of each species of fungi was placed in the 10 ml tube and pulp waste added. The tubes were sealed with a rubber septum (Suba•Seal®) and argon was let in, in order to make the environment anaerobic. Following that, a needle was inserted into the septa as an outlet in order to avoid the build-up of pressure. The tubes were put in a shaker, and held at 27° C. for 24 to 48 h. The amount of ethanol produced was determined by gas chromatography as described above

The results are shown in Tables 5A and B. Significant ethanol production (>2 g/l at 24 h) was recorded for all fungi, except for the control, Penicillium chrysogenum.

It was shown that soft rot fungi Thielavia terrestris produced more ethanol than Trichoderma viridae under these experimental conditions. Similarly, white rot fungi Phanerochaete chrysosporium exhibited the strongest ethanol production capability, 7.96 g/l at 36 hours, under the experimental conditions. Compared to Penicillium chrysogenum, the white rot fungi Phanerochaete chrysosporium produced 5 times more ethanol. Notably, Phanerochaete chrysosporium produced slightly more ethanol than Saccharomyces cerevisiae under the same conditions (7.82 g/l).

TABLE 5A

| | | White rot | Control |
|---|---|---|---|
| | Soft rot | Postia | Penicillium |
| Time | Trichoderma viride | placenta | chrysogenum |
| (h) | (gil) | (g/l) | (g/l) |
| 24 | 2.46 (±0.28) | 2.3 (±0.06) | 1.13 (±1.60) |
| 48 | 2.15 (±0.71) | 1.8 (±0.45) | 1.56 (±0.11) |

TABLE 5B

| | Soft rot | White rot | White rot | |
|---|---|---|---|---|
| | Thielavia | Trametes | Phanerochaete | Brown rot |
| Time | terrestris | versicolor | chrysosporium | Gloeophyllum trabeum |
| (h) | (g/l) | (g/l) | (g/l) | (g/l) |
| 36 | 6.94 | 6.94 | 7.96 | 7.02 |

The conditions were not optimised, however the above tests show that the rot fungi tested were capable of significant ethanol production from pulp waste.

### Example 7

### Identification of Fungi

During the priority year, the inventors have investigated the composition of the mix used in the early experiments, and confirmed the identity of Chalara parvispora, identifying the fungus as AF222473.1 with 100% identity. Further fungi have been identified as Trametes hirsuta (AF516556) with 95% identity, Xylaria sp (AB073534) with 73% identity; and Candida petruhensis (AY585213) with 93% identity. In these experiments, DNA was extracted using a commercial kit (Viogene GG1001 from Techtum AB, Ume\dot{a}, Sweden), amplified and sequenced according to procedures well known in the art.

### Example 8

### Fermentation of Waste Liquors

In further experiments performed during the priority year, the inventors have used fungi samples from CBS (Utrecht, The Nederlands) in the fermentation of waste liquors, and shown that a mix of given fungi result in a synergistic effect (Table 6).

TABLE 6
Fermentation of waste liquor using different microorganisms

| | Ethanol production |
|---|---|
| Sample | (g/l) |
| Control | 0 |
| Saccharomyces cerevisiae | 5.2 +/− 1.03 |
| Chalara parvispora | 7.49 +/− 0.24 |
| Trametes hirsuta | 7.82 +/− 0.87 |
| C. parvispora + T. hirsuta | 17.1 +/− 1.61 |
| C. parvispora + T. hirsuta + S. cerevisiae | 24.1 +/− 1.87 |

It should be noted that the supplier of the waste liquor is capable of producing about 12 g/l ethanol from a liquor containing about 45.5 g hexoses /litre. From the same liquor, the inventors repeatedly produced about 23 to 24 g/l.

Waste liquor has also been subject of further study, and the composition found to vary slightly (Table 7).

TABLE 7
Composition of waste liquor

| | Carbon source (g/l) | | | | |
|---|---|---|---|---|---|
| Sample | Glucose | Mannose | Galactose | Arabinose | Xylose |
| Fresh liquor | 9.7 | 27 | 4.7 | 0.69 | 11 |
| No. 1 | | | | | |
| Fresh liquor | 11 | 29 | 5.0 | 0.61 | 12 |
| No. 2 | | | | | |
| Liquor No. 2 | 6.5 | 19 | 3.6 | 0.39 | 7.6 |
| after 1 week | | | | | |
| storage | | | | | |

The inventors have also made supplementary studies with soft rot, white rot and brown rot fungi, confirming their capability to produce ethanol from waste liquors (Table 8). The tests were performed as in Example 6, with the exception that each fungus was tested in triplicate, and the fermentation time was 40 h.

TABLE 8
Ethanol Produced in waste liquors using rot fungi (ethanol g/l)

| Soft rot fungi | | |
|---|---|---|
| Trichoderma viride | Thielavia terrestris | Postia placenta |
| 8.5 +/− 1.08 | 8.87 +/− 1.13 | 9.57 +/− 1.232 |
| White rot fungi | | |
| Phanerochaete | Trametes versicolor | |
| chrysosporium | 10.47 +− 1.45 | |
| 7.91 +/− 0.08 | | |
| Brown rot fungi | | |
| Gloeophyllum trabeum | | |
| 8.32 +/− 0.73 | | |
| Other fungi | | |
| Chalara parvispora | | |
| 8.84 +/− 0.20 | | |

Although the invention has been described with regard to its preferred embodiments, which constitute the best mode presently known to the inventors, it should be understood that various changes and modifications as would be obvious to one having the ordinary skill in this art may be made without departing from the scope of the invention which is set forth in the claims appended hereto.

1. A process for the production of ethanol through fermentation of organic starting materials, characterized in that at least one fungus belonging to the species Chalara is used, said fungus being capable of metabolizing pentose compounds.

2. The process according to claim 1, wherein said at least one fungus is capable of metabolizing both pentose and hexose compounds.

3. The process according to claim 1, wherein said at least one fungus is used in a mix of fungi, further comprising at least one second fungus belonging to the species Trametes.

4. The process according to claim 1, wherein said at least one fungus is Chalara parvispora.

5. The process according to claim 3, wherein said second fungus is Trametes versicolor.

6. The process according to claim 3, wherein said mix of fungi further comprises one or more fungi chosen among Trichoderma sp., Thielavia sp., Postia sp., Gloeophyllum sp., Phanerochaete sp., or a combination thereof.

7. The process according to claim 3, wherein said mix of fungi comprises the fungi Chalara parvispora, and Trametes versicolor; and at least one of Trichoderma viride, Thielavia terrestris, Postia placenta, Gloeophyllum trabeum, Phanerochaete chrysosporium, or a combination thereof.

8. The process according to claim 1, wherein said fungus or mix of fungi is used in combination with at least one yeast.

9. The process according to claim 1, wherein said at least one fungus or mix of fungi is added prior to, or substantially simultaneously with the addition of the yeast.

10. The process according to claim 8, wherein said yeast is a yeast belonging to the species Saccharomyces.

11. The process according to claim 8, wherein said yeast is Saccharomyces cerevisiae.

12. The process according to claim 1, wherein the fermentation is performed as batch fermentation.

13. The process according to claim 1, wherein the fermentation is performed as a continuous or semi-continuous process, where starting materials and/or nutrients are added during fermentation.

14. The process according to claim l, wherein the pH of the starting material is adjusted to the range of about pH 4.5-7.

15. The process according to claim 14, wherein the pH is adjusted to the range of about 5.5-6.5.

16. The process according to claim 14, wherein the pH is adjusted to about pH 6.

17. The process according to claim 1, wherein the fermentation is performed in a temperature interval of about 20 to about 40° C.

18. The process according to claim 17, wherein the temperature is in the interval of about 26 to about 36° C.

19. The process according to claim 1, wherein the starting material is chosen among:
wood or non-wood plant materials;
waste or by-products from forestry, such as wood chips, saw dust etc;
solid or liquid effluents or by-products from pulp and paper industry, such as wood hydrolysates;
solid or liquid effluents or by-products from food and feed industry, for example, effluents or by-products containing cellulose, hemicellulose, sugar or starch;
solid or liquid waste or by-products from agriculture;
other waste or by-product streams or their components comprising compounds that can be fermented to produce ethanol; and
any of the above-mentioned materials in treated or untreated from.

20. A process for the production of ethanol from a starting material consisting substantially of waste or by-products from forestry, characterized in that at least one fungus belonging to the species Chalara is used, said fungus being capable of metabolising pentose compounds.

21. The process according to claim 20, wherein said at least one fungus is capable of metabolizing both pentose and hexose compounds.

22. The process according to claim 20, wherein said at least one fungus is used in a mix of fungi, said mix further comprising at least one second fungus belonging to the species Trametes.

23. The process according to claim 20, wherein said at least one fungus is Chalara parvispora.

24. The process according to claim 22, wherein said at least one second fungus is Trametes versicolor.

25. The process according to claim 22, wherein said mix of fungi further comprises one or more fungi chosen among Trichoderma sp., Thielavia sp., Postia sp., Gloeophyllum sp., Phanerochaete sp., or a combination thereof.

26. The process according to claim 22, wherein said mix of fungi comprises the fungi Chalaraparvispora and Trametes versicolor, and at least one of Trichoderma viride, Thielavia terrestris, Postia placenta, Gloeophyllum trabeum, Phanerochaete chrysosporium, or a combination thereof.

27. The process according to claim 20, wherein said fungus is used in combination with at least one yeast.

28. The process according to claim 20, wherein the starting material comprises spent liquor (waste liquor) from pulping.

29. A starter culture for use in a process according to any of the above claims, comprising Chalaraparvispora and at least one fungus chosen among Trametes sp., Trichoderma sp., Thielavia sp., Postia sp, Gloeophyllum sp, Phanerochaete sp, or a combination thereof.

30. The starter culture according to claim 29, comprising Chalara parvispora and Trametes versicolor; and at least one of Trichoderma viride, Thielavia terrestris, Postia placenta, Gloeophyllum trabeum, Phanerochaete chrysosporium, or a combination thereof.

31. The starter culture according to claim 29, further comprising a yeast.

32. A growth medium for a fungus used in the process according to claim 1, comprising CaCl₂2H₂O at a final concentration of about 0.0130 g/l, MgSO₄ 7H₂O at a final concentration of about 0.030 g/l, K₂HPO₄ at a final concentration of about 0.95 g/l, NaH₂PO₄ 2H₂0 at a final concentration of about 0.80 g/l, D-xylose at a final concentration of about 25 g/l, D-mannose at a final concentration of about 25 g/l, D-galactose at a final concentration of about 25 g/l, NH₄Cl at a final concentration about 0.5 g/l, and salts at a final concentration about 0.040 g/l.

33. The growth medium according to claim 32, further comprising starch at a final concentration of about 25 g/l.
